(19) United States
(12) Patent Application Publication
(10) Pub. No.: US 2021/0161394 A1
(43) Pub. Date: Jun. 3, 2021

(54) SYSTEM, METHOD, AND COMPUTER-ACCESSIBLE MEDIUM FOR NON-INVASIVE TEMPERATURE ESTIMATION

(71) Applicant: The Trustees of Columbia University in the City of New York, New York, NY (US)

(72) Inventors: Sairam GEETHANATH, New York, NY (US); Julie Marie KABIL, New York, NY (US); John Thomas Vaughan, Jr., New York, NY (US)

(73) Assignee: The Trustees of Columbia University in the City of New York

(21) Appl. No.: 17/170,214

(22) Filed: Feb. 8, 2021

Related U.S. Application Data
(63) Continuation of application No. PCT/US2019/046172, filed on Aug. 12, 2019.
(60) Provisional application No. 62/717,858, filed on Aug. 12, 2018.

(51) Int. Cl. A61B 5/01; A61B 5/055; G06T 7/00; G06N 3/08

(52) U.S. Cl. CPC A61B 5/015; A61B 5/055; G06T 2207/30024; G06N 3/08; G06T 2207/10088; G06T 7/0012

(57) ABSTRACT

Exemplary system, method and computer-accessible medium for estimating a temperature on a portion of a body of an anatomical structure(s) can be provided, using which it is possible to, for example, receive a plurality of magnetic resonance (MR) images for the anatomical structure(s), segment the MR images into a plurality of tissue types, mapping the tissue types to a tissue property(ies), and estimate the temperature on the portion of the body of the patient(s) using a neural network. The tissue property(ies) can include a conductivity, a permittivity, or a density. The density can be a mass cell density. The neural network can be a single neural network. The temperature can be estimated based on a set of vectors between points on the portion of the body and a temperature sensor. Each vector can correspond to a tissue thermal profile for each point.

### CROSS-REFERENCE TO RELATED APPLICATIONS

This application relates to and claims priority from U.S. Patent Application No. 62/717,858, filed on Aug. 12, 2018, the entire disclosure of which is incorporated herein by reference.

### FIELD OF THE DISCLOSURE

The present disclosure relates generally to temperature estimation, and more specifically, to exemplary embodiments of an exemplary system, method, and computer-accessible medium for non-invasive temperature estimation.

### BACKGROUND INFORMATION

Standardized temperature measurement has a history of more than 600 years. However, non-invasive temperature measurement of samples still remains a challenge. Temperature probes of diverse kinds ranging from optic fiber probes, thermocouples, thermistors to thermochromic materials are commercially available for measurement. These sensors rely on the methods of transduction of converting a measure of heat into an electrical or calorimetric property such as electrical resistance or change in absorbed reflected visible light. However, these sensors are limited by their ability to measure temperature in the vicinity of the sensor. In particular, external temperature sensors provide highly accurate but highly localized measurements proximal to the sensor. In contrast, commercial applications requiring knowledge of temperature variations and thermal stresses, such as those experienced in clinical procedures, material sciences, pharmaceutical drug delivery systems, power optimization studies, etc. require spatial mapping of temperature inside of the samples.

These samples are typically subjected to specific experimental conditions that further compound internal measurement of temperature. These challenges have necessitated the development of temperature estimation methods based on indirect and/or reference assessments. These methods typically employ the electromagnetic spectrum to interrogate sample characteristics exploited through imaging physics.

Exemplary Infrared Thermal Imaging

Thermal imaging and regular imaging have some similarities. While a regular camera can generate images using the visible part of the electromagnetic spectrum (e.g.,. with a wavelength λ between 380 nm and 780 nm), infrared thermal imaging systems can use a specific infrared part of the electromagnetic spectrum (e.g., being between 7 μm and 14 μm) to generate thermal images. Infrared thermal imaging is based on the principle that every object emits infrared radiation, the amount of which can depend on its temperature. Infrared thermal cameras use sensors which are dependent on the infrared radiation they absorb, which can then facilitate the generation of the thermal images with an accuracy as high as about ±1%±1° C. and a thermal sensitivity as high as about 0.03 ° C.

Therefore, although thermal imaging can be beneficial in various situations and especially for finding hot spots, its accuracy may not be always be sufficient for human thermal safety applications if precise, absolute, values are needed.

### Electrical Impedance Tomography (“EIT”)

Another approach can be the imaging of conductivity of different tissues by applying electric currents at the surface of the body using electrodes and measuring the electric potentials. As tissue temperature and tissue conductivity can be directly related (e.g., 2% per ° C), this modality can be used to measure temperature. However, the FIT procedure can have some drawbacks, such as a decreased sensitivity for the tissue away from the electrodes, leading to poor spatial resolution and insufficient accuracy.

MREIT (e.g., Magnetic Resonance Imaging combined with EIT) modalities have shown some promising results to help tackle these problems, but more research has to be performed to conclude on the usefulness of EIT for thermal imaging.

### Exemplary MR Thermometry

### Exemplary Electromagnetic And Thermal Simulations

To tackle some of the challenges posed by non-invasive temperature measurements, electromagnetic and thermal simulations can be a method that could be widely used. For example, the electromagnetic and thermal simulations can be based on solving Maxwell's equations and on the Pennes' Bioheat equation. Several numerical human models were developed during the past decades, which can be used in numerical simulations to obtain Specific Absorption Rate (“SAR”) and temperature maps resulting from electromagnetic exposure. However, the models and settings used for simulation should be as accurate as possible to correctly reflect reality. Further, due to the huge variability between different human anatomies, the SAR and temperature maps cannot be extrapolated from one model to another, thus the need for personalized models exists.

### Exemplary Thermometry With T₁/Spin-Lattice Relaxations Time

One of the first methods of magnetic resonance (“MR”) thermometry was the use of the characteristic time T₁, which can depend on the interactions between molecules and thus can be directly affected by temperature changes. Below is one exemplary model describing this relation:

$${T}_{1} ∝ {e}^{- \frac{{E}_{a} ( {T}_{1} )}{kT}}$$

Eₐ(T₁) can be the activation energy of the relaxation process, k can be the Boltzmann constant and T can be the absolute temperature.

Research on T₁-based thermal is has shown that even if the link between and temperature can be well established with an average temperature-dependence of 1%/° C., it can be a time-consuming method and can also be compromised by the lack of knowledge of the exact thermal properties of the tissues. For these reasons, T₁ can preferably be used to get a qualitative information on temperature distribution rather than accurate quantitative measurements.

### Exemplary Proton Resonant Frequency Shift (“PRP”)

The PRF method is another MR thermometry method. This method is based on the link existing between the proton shielding by the electrons (e.g., which can impact the resonance frequency) and the temperature. Water molecules can be bound by hydrogen bonds. When temperature increases, these bonds can break, and the electrons can shield the proton, causing a shift in the resonance frequency f. With ω=2πf, B₀ being the intensity of the static magnetic field, γ being the gyromagnetic ratio and σ being the screening constant:

ω=γB₀(1−σ)

The linear temperature dependency is described as, for example:

σ(T)=aT

This widely used method is however, limited by field inhomogeneity and motion, two parameters that can decrease the accuracy.

### Exemplary Diffusion Weighted Imaging (“DWI”) Thermometry

Another specific MR thermometry procedure has been developed. Such procedure uses the relation existing between the water diffusion coefficient D and temperature. Using the same notation as in (ii), this can be described as follows:

$$D \approx {e}^{- \frac{{E}_{a} ( D )}{kT}}$$

MRI systems and procedures can measure the diffusion coefficients by the attenuation of MR signals, it can be possible to measure temperature this way with this highly temperature-sensitive method. However, without an additional accelerating and motion-sensitivity targeted procedure, this method can be time consuming, and its result can be compromised by motion. Moreover, the complexity of in-vivo tissue and their variability in thermal properties associated with heat-induced changes in diffusion coefficients can make this method complicated.

### Exemplary Chemical Exchange Saturation Transfer (“CEST”)

Other MR thermometry systems and procedures use temperature-sensitive contrast agents. For the CEST method, the bulk water signal intensity can be measured in response to the presence of a paramagnetic lanthanide complex. The signal intensity and the temperature can be linked by the exchange rate via Arrhenius' law. Thus, tier example:

$$k = A {e}^{\frac{- {E}_{a}}{{k}_{B} T}}$$

With k being the rate constant, A being the pre-exponential factor, E being the activation energy of the chemical reaction, T being the absolute temperature in Kelvin, and k_{B} being the Boltzmann constant. Therefore, the agent concentration, if known, can facilitate temperature measurement. Some drawbacks of this method include the necessity of knowing exactly the agent concentration, which can be difficult. Moreover, the imaging time can be long.

Thus, it may be beneficial to provide exemplary system, method, and computer-accessible medium for non-invasive temperature estimation which can overcome at least some of the deficiencies described herein above.

### SUMMARY OF EXEMPLARY EMBODIMENTS

Exemplary system, method and computer-accessible medium for estimating a temperature on a portion of a body of an anatomical structure(s) can be provided, using which it is possible to, for example, receive a plurality of magnetic resonance (MR) images for the patient(s), segment the MR images into a plurality of types, mapping the tissue types to a tissue property(ies), and estimate the temperature on the portion of an anatomical structure(s) using a neural network. The tissue property (ies) can include a conductivity, a permittivity, or a density. The density can be a mass cell density. The neural network can be a single neural network. The temperature can be estimated based on a set of vectors between points on the portion of the body and a temperature sensor. Each vector can correspond to a tissue thermal profile for each respective point.

In certain exemplary embodiments of the present disclosure, the temperature can be mapped at each respective point, which can be performed using the neural network and/or a Euclidean distance between each point and the temperature sensor. The portion of the body can be on a surface of the anatomical structure(s) or internal to the anatomical structure(s). The tissue types can include at least one of (i) Fat, (ii) Grey Matter, (iii) Bone, (iv) Muscle, and (iv) Cerebrospinal Fluid. The neural network can be trained, for example, by segmenting the tissue types of at least one further anatomical structure and/or by varying, a number of hidden nodes in the neural network. The neural network can include, e.g., (i) three layers, and (ii) a Rectified linear Unit activation function. The anatomical structure(s) can be a brain of a patient, and the MR images can be brain slices of the brain of the patient.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1A is an exemplary diagram illustrating an exemplary human numerical model inside a head birdcage coil according to an exemplary embodiment of the present disclosure;

FIGS. 1B and 1C are exemplary images generated by having polygons drawn around different brain areas according to an exemplary embodiment of the present disclosure;

FIG. 1D is an exemplary permittivity map according to an exemplary embodiment of the present disclosure;

FIG. 1E is an exemplary conductivity map according to an exemplary embodiment of the present disclosure;

FIG. 1F is an exemplary density map according to an exemplary embodiment of the present disclosure;

FIG. 2A is an exemplary graph of the temperature estimated using non-invasive temperature estimation temperatures compared to CST results according to an exemplary embodiment of the present disclosure;

FIG. 2B is an exemplary scatter plot of the temperature of all of the points in a slice test according to an exemplary embodiment of the present disclosure;

FIG. 2C is an exemplary graph of the temperature estimated using the non-invasive temperature estimation and CST simulated data for a test slice according to an exemplary embodiment of the present disclosure;

FIG. 2D is a simulated CST temperature map for a training slice;

FIG. 2E is a simulated temperature map for a test slice according to an exemplary embodiment of the present disclosure;

FIG. 2F is an estimated temperature map generated using the non-invasive temperature estimation for a test slice according to an exemplary embodiment of the present disclosure;

FIG. 3A is an exemplary illustration of an exemplary adult male model according to an exemplary embodiment of the present disclosure;

FIG. 3B is an exemplary brain slice temperature map according to an exemplary embodiment of the present disclosure;

FIG. 4A is another exemplary permittivity map according to a further exemplary embodiment of the present disclosure;

FIG. 4B is another exemplary conductivity map according to a further exemplary embodiment of the present disclosure;

FIG. 4C is another exemplary density map according to a further exemplary embodiment of the present disclosure;

FIGS. 5A and 5B are exemplary correlation graphs between the real and predicted temperature according to an exemplary embodiment of the present disclosure;

FIG. 5C is an exemplary graph illustrating the cost versus the number of iterations according to an exemplary embodiment of the present disclosure;

FIGS. 6A and 6C are exemplary temperature maps obtained with CST according to an exemplary embodiment of the present disclosure;

FIGS. 6B and 6D are exemplary temperature maps predicted by the exemplary NITE according to an exemplary embodiment of the present disclosure;

FIG. 7 is an exemplary flow diagram of a method 800 for estimating a temperature of a portion of a body of an anatomical structure according to an exemplary embodiment of the present disclosure; and

FIG. 8 is an illustration of an exemplary block diagram of an exemplary system in accordance with certain exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

### Exemplary Non-Invasive Temperature Estimation

For example, an object O (\vec{r}) can be provided with an internal spatial temperature distribution T(\vec{r}) with \vec{r} representing the three dimensional spatial vector r(x, y, and z). Let T(\vec{r}) be altered through the application of an external heating source such as the application of radio frequency pulses during a magnetic resonance imaging (“MRI”) experiment. This can cause changes in T(r) depending on a multitude of factors. Some examples of such factors can include the characteristics of the heat source, material composition of the object relating to corresponding electro-thermal properties, position of the object, capabilities of the object to regulate the changes in temperature, etc. In the case of in vivo studies utilizing MRI, this can map to radio frequency (“RF”) Transmitter (\vec{θ}_{RFT}) and Pulse Sequence Design (\vec{θ}_{PSD}) parameters, tissue thermal properties (\vec{θ}_{TP}), posture (\vec{θ}_{POS}) thermos-physiological regulation (\vec{θ}_{Tphy}) capabilities, etc. Thus, for example:

T(\vec{r})=f(\vec{θ})

\vec{θ}=f(\vec{θ}_{RFT}, \vec{θ}_{PSD}, \vec{θ}_{TP}, \vec{θ}_{POS}, \vec{θ}_{Tphy}, . . . ) [1]

The surface temperature (T_{S}(\vec{r})) of the object can be measured through one of the exemplary modalities described herein. The number of these sensors can be Nₛ. The temperature changes in these measurements can be caused by the internal heat changes in the object and the heat source.

There can be N_{P} points inside O (\vec{r}) whose temperatures have to be estimated. For a point Q (T_{Q}(\vec{r})) inside of O(\vec{r}), there can be Nₛ observations of surface temperatures (T_{S}(\vec{r})). Changes in the temperature at point Q can cause changes (e.g., widely ranging from subtle to significant) in T_{S}(\vec{r}). Thus, for example:

T_{S}(\vec{r})=f(T_{Q}(\vec{r})) [2]

These Nₛ observations can also be impacted by changes in the other (N_{P}−1) points inside O (\vec{r}. Now consider a set of vectors between point Q and the temperature sensors T_{S}(\vec{r}). MR images of the object can be included. These can be segmented into different tissue types and subsequently mapped to tissue/material properties such as conductivity, permittivity, mass cell density, etc. Each of the N_{P} points can now have associated vectors corresponding to tissue thermal profiles (P_{N}_{_{P}}(\vec{r})) relating them to Nₛ sensors. For example, the tissue thermal profile vector relating point Q with one of the temperature sensors Tₛ₁(\vec{r}) can be, for example:

P_{Q-T}_{ₛ₁}(\vec{r})=[ε(\vec{r}), σ(\vec{r}), ρ(\vec{r}), ∥Q−Tₛ₁∥₂, Tₛ₁(\vec{r})] [3]

This can then be cast as a neural network based inverse problem of mapping temperature \hat{T}_{N}_{_{P}}(\vec{r}) at each of the N_{P} points in O(\vec{r}). This can be derived from a given set of tissue thermal properties deduced from MR images of the object, the Euclidean distance between the point and each of the Nₛ sensors, and the measured temperature T_{S}(\vec{r}) by each of the Nₛ sensors. Thus, for example:

\hat{T}_{N}_{_{P}}(\vec{r})=f(P_{N}_{_{P}}(\vec{r})) [4]

The exemplary formulation can be simplified by converting the problem from that of regression to multi-class classification. This can be possible in the context of in vivo human imaging due to the well-defined range of temperature (e.g., 37° C.-41° C.) as well as a precision of 0.1° C. that can be beneficial for applications dependent on temperature estimation. This can define the number of labels (k) for the formulation. The operator f (.) can be evaluated by the neural network. In relation to existing thermal solvers, this can correspond to the joint estimation of SAR and temperature maps while including two additional inputs of the normal metric and surface temperatures. Thus, for example:

\dot{\hat{T}}_{N}_{_{P}}(\vec{r})=f(P_{N}_{_{P}}(\vec{r}) [5]

where, for example:

\dot{\hat{T}}_{N}_{_{P}}(\vec{r})=kΔ\hat{T}_{N}_{_{P}}(\vec{r}) [6]

The training phase can then include the actual temperature at point Q as an input in addition to the thermal tissue profile vector (P_{Q-T}_{ₛ₁}(\vec{r})) as shown in Eq. 3. The testing can be performed through the computation of Eq. 5

It can be beneficial to non-invasively estimate the temperature of the internal portions of an object through the training and testing of a fast, efficient neural network as a multi-class classification supervised learning problem. In particular, these can correlate well with state-of-the art thermal solvers that can be routinely used for simulations and temperature related downstream decisions in MRI.

### Exemplary Methods

Application to MR based Thermometry Simulations

The formulation was tailored to provide in vivo temperature maps under the influence of radio frequency pulses at 3T. This included generation of the thermal vectors, internal and surface temperatures for a human model using CST. This data was then utilized by the neural network separately for training and testing.

### Exemplary CST Simulations

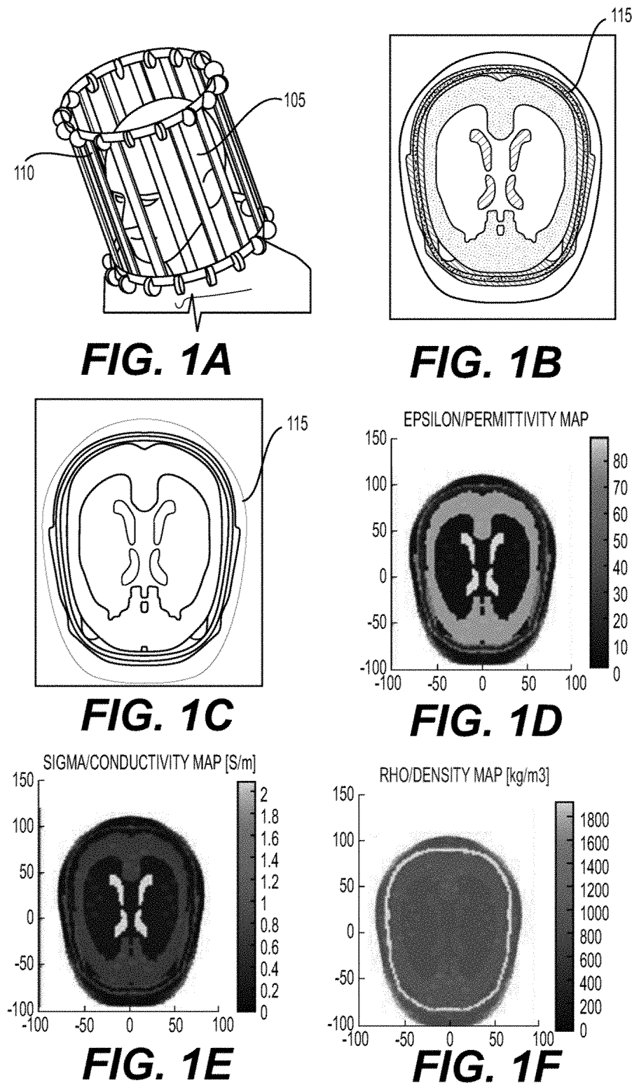

The simulations to acquire temperature maps were performed using the CST Studio Suite software 2018 (e.g., CST Darmstadt, Germany). The procedure step was to model a 16 rung birdcage head coil and tune it to 128 MHz, by adjusting its capacitance values, using a circular polarization to have a homogeneous RF field. Then, a human numerical model, Tom, was imported. (See e.g., diagram shown in FIG. 1A). For example, FIG. 1A shows an exemplary diagram illustrating a human numerical model 105 inside a bead birdcage coil 110 according to an exemplary embodiment of the present disclosure. The exemplary diagram included a voxel model and the resolution used was 2 mm×2 mm×2 mm. This numerical model had 30 different tissue types which were all attributed their own material properties (e.g., mechanical, dielectric, thermal). Additional simulations were performed using a 4T TEM head coil at 170 MHz. with a training model and testing model. A voxel size of 0.08×0.08×0.08 mm³ was used. The output includes thermal maps for 3 brain slices on the training model and 1 brain slice on the testing model.

Once the exemplary model was set up, an exemplary mesh (e.g,, more than 16 million cells) was used to ensure the accuracy of the results. Then, the electromagnetic simulation using the Time-Domain solver was performed and completed in approximately 10 hours. The electromagnetic simulation results wore then provided to the thermal simulation which used the Pennes' Bioheat equation. The Thermal transient solver was performed for an about 100 seconds-duration and the temperature was recorded using a three dimensional (“3D”) monitor with a recording step of 10 seconds. The thermal simulation took about 15 minutes to complete. The thermal maps were obtained at about 100 seconds and extracted temperature data from different brain slices, for training and test purposes.

### Exemplary CST Data Processing

FIGS. 1B and 1C show exemplary images generated by having polygons 115, 120, respectively, drawn around different brain areas according to an exemplary embodiment of the present disclosure. FIG. 1D shows an exemplary permittivity map according to an exemplary embodiment of the present disclosure. FIG. 1E shows an exemplary conductivity map according to an exemplary embodiment of the present disclosure. FIG. 1F shows an exemplary density map according to an exemplary embodiment of the present disclosure. Three input temperature maps were used, and the brain slices considered for the exemplary non-invasive temperature estimation (“NITE”) implementation were segmented. into five tissue types: Fat, Grey Matter, Bone, Muscle, and Cerebrospinal Fluid. These segments were then assigned corresponding tissue properties using MATLAB (e.g.. The Mathworks Inc., MA). This operation was performed, by drawing polygons around the different brain areas. (See e.g., polygons 115 shown in FIGS. 1B and 1C). Tissue properties maps were generated after segmentation of the numerical human model brain slice in CST and after processing in Matlab. For example, each tissue property (e.g., conductivity, permittivity and density) was then to the corresponding polygon, facilitating the generation of a permittivity map (see e.g., permittivity map shown in FIG. 1D), a conductivity map (see e.g., conductivity map shown in FIG. 1E) and a density map (see e.g., density map shown in FIG. 1F). These exemplary tissue properties maps facilitate the correct tissue properties attribution to each point inside the brain slice. The tissue property values on these maps can be attributed to the numerical human model itself, prior to running the exemplary Electromagnetic and Thermal simulations. The training and test matrices were generated and fed to the machine learning procedures.

### Exemplary Neural Network Architecture

Exemplary neural network architecture was selected based on the following exemplary features:

Exemplary Rapid inference: It can be beneficial to facilitate near real-time computation of the temperature maps given that the whole body MRI data approximately consists of 16 million points (e.g., Np) for a resolution of 2 mm×2 mm×2 mm. This resulted in the need of shallow networks.

Exemplary True multi-class classification: It was beneficial to use a hybrid binary and multi-class classification network to enable a variable number of classes and temperature increments.

Exemplary Limited number of tunable hyper-parameters: The chosen network can be limited to facilitate simplicity ease-of-use, reproducibility and robustness

These desired features resulted in the choice of Extreme Learning Machines (“ELM”) as the neural network architecture for NITS. An ELM implemented on tensorflow was utilized for demonstration of the formulation. For example, the only tunable hyper-parameter in ELMS can be the number of hidden nodes. The number of nodes was varied from 512 to 2560 to determine validation test accuracy. For this implementation, 2048 hidden nodes (e.g., corresponding to the highest attained accuracy of 82%) with sigmoid as the activation function and with softmax providing the utilized probabilities for multi-class classification were chosen.

### Exemplary Input and Validation Data Features and Training

The input data for training included 6690 examples derived from one slice of the brain CST simulations. The validation set was chosen to be 134 examples corresponding to 2% of the training data. The chosen training data was the slice with the highest temperature range (e.g., 37.1 . . . 39.8° C.). This was to ensure that the network can be aware of the full range of temperatures it was likely to see during testing. The tissue thermal vector (P_{N}_{_{P}}(\vec{r})) included 11 discrete points regardless of the distance between the point and the surface temperature sensors. Each row of the training included 35 features (e.g., 33 for the thermal properties, norm of the distance and the actual temperature at the point Q). Each column of this input data matrix was resealed to unit range. The data was further modified by adding random uniform noise with varying levels of intensity (e.g., 0.1 to 0.3 in steps of 0.05). Each point Q had four observations (Nₛ) of these features with each corresponding to one surface temperature sensor measurement.

### Exemplary Training Labels (Ground Truth)

The temperature range was divided into segments each separated by 0.1° C. This resulted in 28 bins for the temperature range. This vector was stored for translation between actual temperatures and the labels (e.g., indices of the vector) for training.

### Exemplary Input Data-Testing

The trained network was saved as a model and utilized for testing. A slice of the brain about 5 mm from the training slice was utilized for testing. All points in each of the two dimensional slice along with the corresponding features were flattened and reshaped to the dimensions similar to the ones described for the training data. The resulting labels were converted to temperature bins each of width 0.1° C.

### Exemplary Error Quantitation and Statistical Analysis

Training and test results were correlated with CST simulations. The temperatures from CST simulations were binned correspondingly to enable discrete comparisons between the two approaches. A paired t-test was performed to evaluate statistical significance. All statistical evaluations were performed using graphpad Prism.

### Exemplary Computational Resources and Performance

All CST computations performed using the Time-Domain solver was accelerated by a graphics processing unit (“GPU”). These calculations were performed with a workstation equipped with 4 Nvidia Tesla K-80 cards. The neural network implementation was performed on a custom Digital Storm computer with i9 Intel Processor, and 4 NVidia GPU Tesla cards of 12 GB each. The total time for training over 5 trials was tabulated. The testing/inference performance for each of the N slices was recorded.

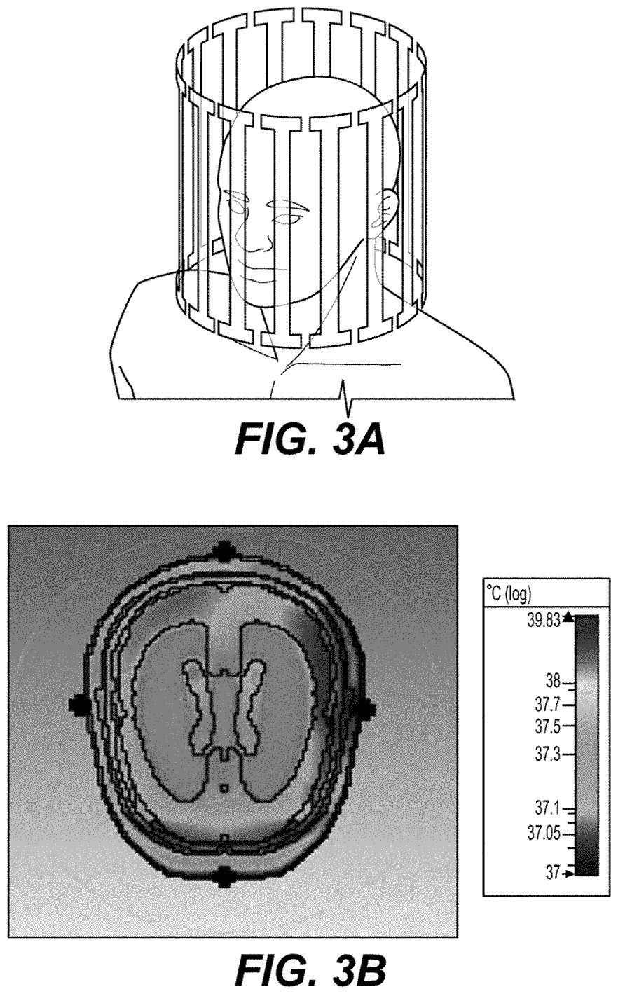

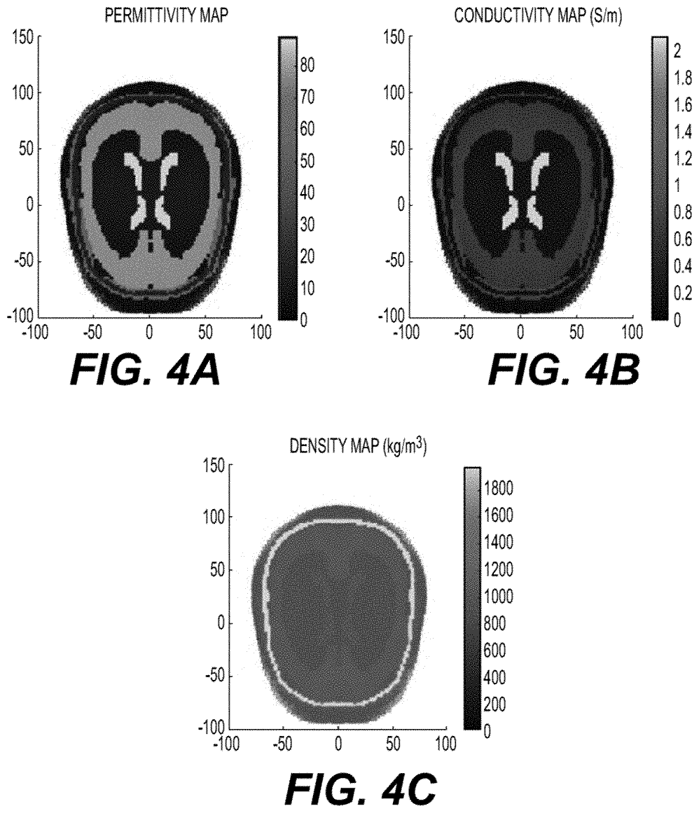

An electromagnetic/thermal co-simulation was performed using CST (e.g., Dassault Systèmes, France). A 16 rungs 3T birdcage head coil was modeled. A human numerical model (e.g., “Tom”, adult male from the CST Voxel Family) was imported in the head coil with a 2 mm isotropic resolution. (See, e.g., exemplary diagram shown in FIG. 3A). A time-domain electromagnetic simulation was performed with the coil tuned to 123 MHz. Then, the thermal losses were used to perform a 100 seconds-long thermal simulation. The temperature for three different axial slices of the brain, at z-20 mm, z=23 mm and z=35 mm, was saved at 100 seconds. (See, e.g., exemplary map shown in FIG. 3B). The slices were manually segmented in CST with five different tissue types (e.g., Fat, Gray Matter, Bone, Cerebrospinal Fluid, Muscle) and then imported in MATLAB to obtain the following properties maps for each slice: an exemplary permittivity map (e.g., exemplary map shown in FIG. 4A), an exemplary conductivity map (e.g, map shown in FIG. 4B), and an exemplary density map (e.g., exemplary map shown in FIG. 4C).

The exemplary (e.g., thermal) maps shown in FIGS. 4A-4C were also imported in MATLAB for the deep teaming implementations. The exemplary neural network, implemented with TensorFlow, was trained using 6690 points from the brain slice that had the highest temperature range among the three slices selected (e.g., 37.1° C.-39.8° C.), for example, the z=20 mm slice. The neural network had 3 layers, 2048 nodes, a Rectified Linear Unit (“ReLU”) activation function, a learning rate of 0.0001, 6000 epochs and 46 labels. The cost after the final epoch was 0.39. The input included 3 training slice with 191,866 training points. Two of the training slices had 155,204 training points, with 2% of the training set used for validation. The exemplary goal was to overfit before optimizing.

The input was a matrix with 35 features for each point: 33 tissue properties (e.g., 3 properties for each of the 11 segments), 1 norm distance to one of the four sensors, 1 temperature value. Each point was represented 4 times to consider the distance and properties to the 4 sensors. Random Gaussian noise with standard deviation 0.05 was added to provide for data augmentation. The exemplary hyper-parameters were optimized to improve the accuracy while avoiding over-fitting the model to the training slice. The estimated temperature maps for test slices were plotted, compared to the simulated maps and the correlation between the prediction and the true temperature was assessed.

### Exemplary Results

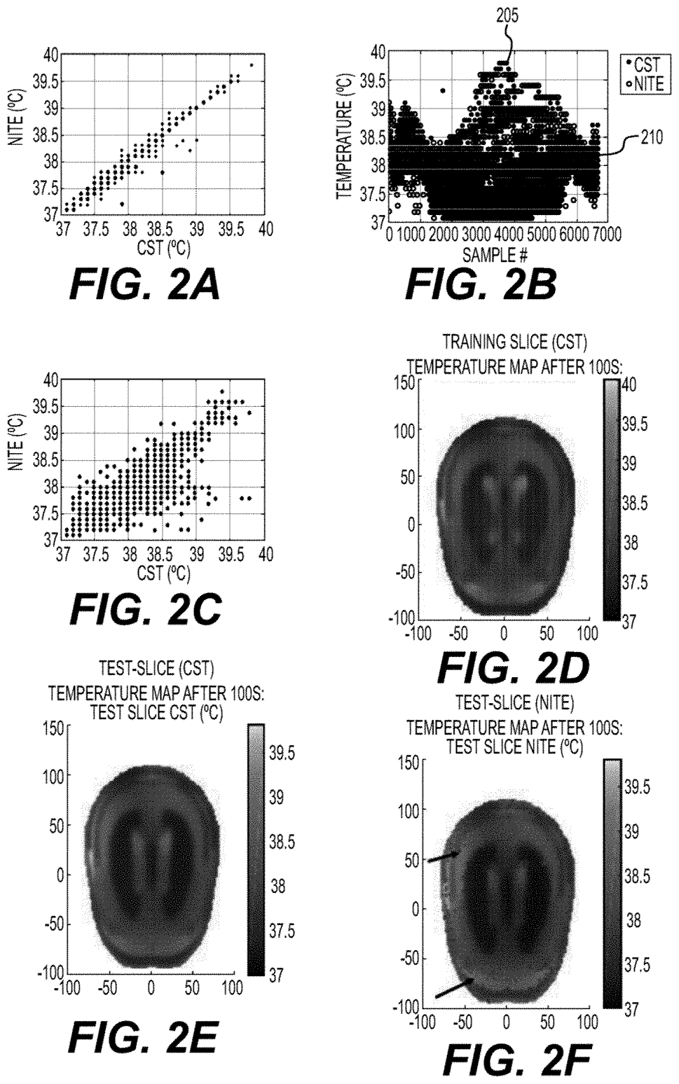

FIGS. 2A-2F show exemplary results of the exemplary NITE. The exemplary graph shown in FIG. 2A depicts a significant positive correlation between the CST simulations (e.g., ground truth) and the validation data set. This indicates the ability of the exemplary neural network to converge with well-balanced bias-variance errors. An accuracy of 82% was deemed acceptable to facilitate for generalization while applying NITE to adjacent slices. FIG. 2B illustrates an exemplary point-wise distribution of the temperatures resulting from CST and NITE for the test slice. It can be observed that the CST (element 205) and NITE (element 210) can be closely matched. FIG. 2C shows an exemplary positive correlation of NITE with the CST data for the test slice. FIGS. 2D-2F show exemplary spatial distribution(s) of temperature corresponding to the training slice, CST simulations for the test slice and NITE temperature maps. The computation time for a slice in CST with the described computational resources was 10.25 hours for the whole body while NITE computed the maps were approximately 250 ms for 1 slice.

For this phantom with a height of 174 cm, the total NITE computation time at resolution of 2 mm resulted in 870 slices. This was computed by NITE in 3.7 minutes without the use of parallelization through GPU. This acceleration was based on the rapid inferencing of the ELMS. Also, the results illustrate how the exemplary NITE can non-invasively map temperature learning from surface temperature and tissue thermal properties (e.g., MR images). This can be validated by the qualitative comparison of FIGS. 2C, 2E, and 2F, and the corresponding estimated temperature ranges resulting from the two approaches for the test slice. Further, the test slice and training slice can be visibly different in the spatial distribution of tissues. This indicates the exemplary NITE's ability to learn variations in geometry.

The exemplary system, method, and computer-accessible medium, according to an exemplary embodiment of the present disclosure, may not need to utilize invasive temperature measurement probes leading to challenges related to measurement and the sample. Accelerated temperature estimation can be achieved due to the utilization of deep learning rather than Finite Difference Time Domain or Frequency Domain simulations that can be computationally expensive. The exemplary system, method, and computer-accessible medium, according to an exemplary embodiment of the present disclosure, can utilize thermal simulation data, sample composition, and surface temperature measurements to compute internal temperature map estimates. For temperature estimation of MR subjects, the exemplary system, method, and computer-accessible medium may only utilize images as compared to other methods that specifically utilize MR thermometry based acquisition methods to be performed. For non-destructive testing, the exemplary system, method, and computer-accessible medium can utilize information from the material properties of the sample and surface temperature data to provide temperature estimates underutilized experimental conditions such as but not limited to direct heating, thermal ablation, etc.

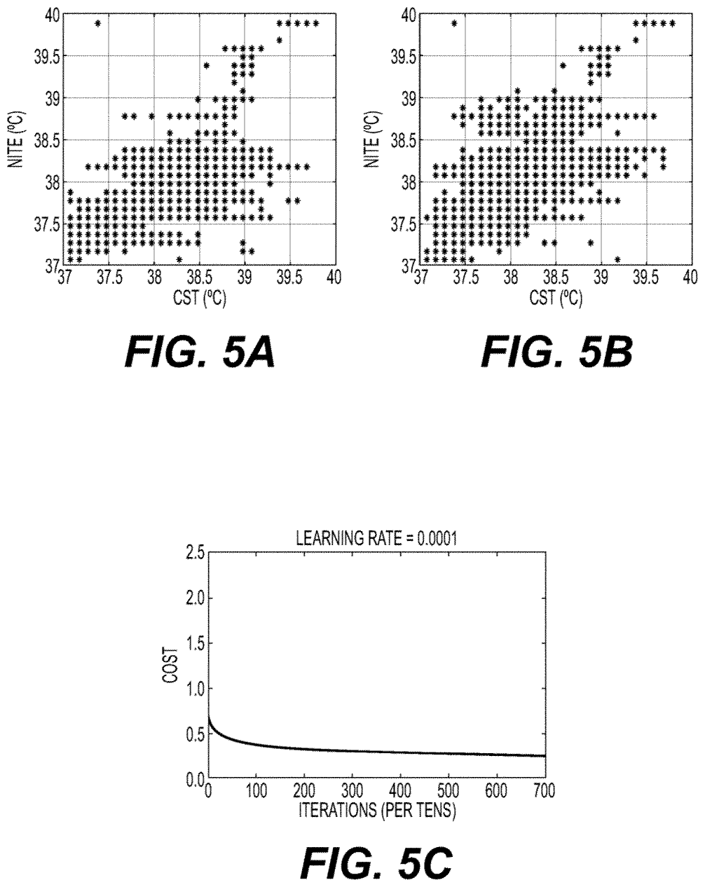

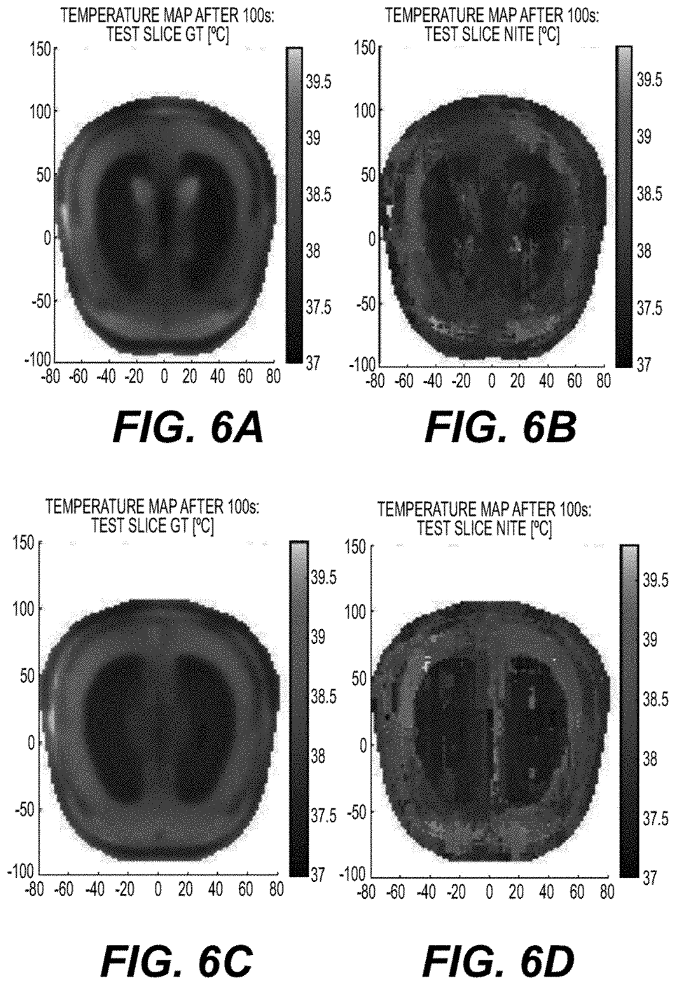

FIG. 5A and 5B show exemplary correlation graphs between the real and predicted temperature, and FIG. 5C shows an exemplary graph illustrating the cost versus the number of iterations. For the graph shown in FIG. 5A, slice z23, for the graph shown in FIG. 5B, slice z=35. FIGS. 6A-6D illustrate further exemplary temperature maps obtained with CST (see, e.g., FIGS. 6A and 6C) and exemplary maps predicted by exemplary NITE (see, e.g., FIGS. 6B and 6D). For example, FIG. 6A shows an exemplary real temperature map, or ground truth, for the z=23 slice, and FIG. 6B shows another exemplary predicted temperature using the exemplary NITE for the z=23 slice. FIG. 6C shows an exemplary real temperature map, or ground. truth, for the z=35 slice, and FIG. 6D shows a further exemplary predicted temperature using the exemplary NITE for the z=35 slice.

An accuracy of 86% was obtained for the training by adjusting the neural network parameters and using a random dropout method. A positive, linear correlation was observed. However, given the structural differences between the training slice and the test slices, the linearity may not yet be optimal: the further away from the training slice, the less accurate the prediction becomes. Table 1 below shows an illustration of a comparison of time performance between the exemplary NITE and CST for the whole body for CST, and one brain slice for NITE after training. For example, 870 slices may be beneficially used for the whole body with a resolution of 2 mm, resulting in an approximate computation time of 2.5 minutes for the exemplary NITE. Once trained, the exemplary neural network can generate the maps several orders of magnitude faster. The exemplary system, method and computer-accessible medium, according to an exemplary embodiment of the present disclosure, can be modified by tuning more finely the random dropout, optimizing the time or adjusting the number of nodes, and afterwards testing on brain slices with bigger structural differences. Alternatively or in addition, a brain volume (e.g., TOM's) can be trained and tested on other models such as DUKE. Then, an in-vitro review can be performed on a phantom with surface and internal temperature sensors to validate the exemplary method experimentally.

TABLE 1

| CST total simulation time: Whole Body | 10.25 | hours |
|---|---|---|
| NITE total calculation time: training | Training (to do once): 1.6 hours | |
| and testing on 1 slice | Testing: 172 ms per slice | |
| NITE estimate total calculation time: | 2.5 | minutes |
| Test on Whole Body | | |
| 870 slices with a resolution of 2 mm | | |

Time-efficiency and radiofrequency safety can be beneficial in MRI protocols. Although MR Thermometry procedures exist, including for example T1 relaxation and proton resonance frequency shift, their sensitivity or their acquisition time can be prohibitive. (See, e.g., Reference 1). The exemplary system, method and computer-accessible medium according to an exemplary embodiment of the present disclosure can include a personalized, non-invasive approach. For example, knowing the tissue properties and distance to several surface temperature sensors and the surface temperature, the exemplary system, method and computer-accessible medium can be used to accurately predict the internal body temperature. In a brain slice, e.g., N points can be considered where it can be beneficial to know/determine the temperature. Thus, N₂ surface temperature sensors can be considered/analyzed. For each point N_{P} in N, an additional set of i equidistant points placed on an imaginary line between one surface sensor and N_{P} can be considered/analyzed. Using MR1 procedure(s), the images can be acquired and segmented to attribute to each point it tissue properties. Considering this as a classification problem with a defined precision, for example 0.1° C., a neural network model can be trained on a numerical brain slice with multiple points using their attributes (e.g., tissue properties, distance to tour surface sensors and known temperatures acquired through simulation). This exemplary model and/or the exemplary attributes can then be tested on two other slices and compared with the real values to estimate the accuracy.

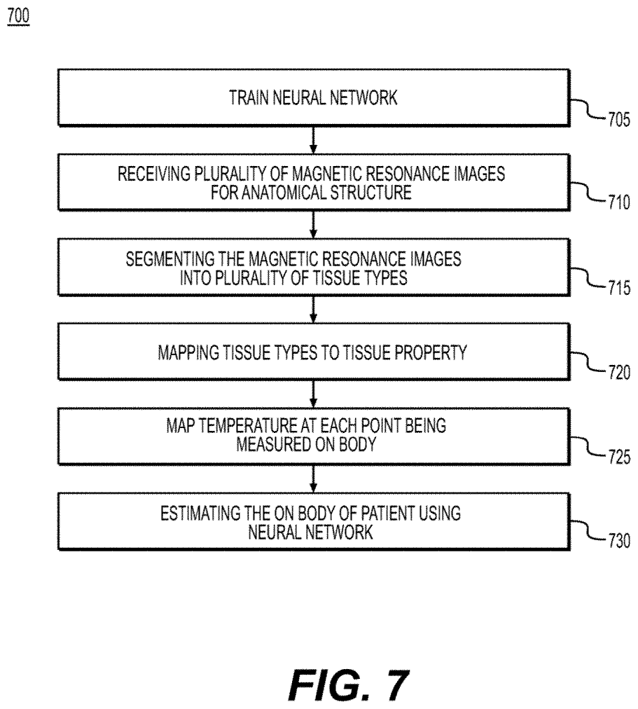

FIG. 7 shows an exemplary flow diagram of a method 700 for estimating a temperature on a portion of a body of an anatomical structure according to an exemplary embodiment of the present disclosure. For example, at procedure 705, a neural network can be trained (e.g., based on the training procedures described above). At procedure 710, a plurality of magnetic resonance (MR) images for the anatomical structure can be received. At procedure 715, the MR images can be segmented into a plurality of tissue types. At 720, the tissue types can be mapped to one or more tissue properties. At procedure 725, the temperature at each point being measured on the body can be mapped. At procedure 730, the temperature on the body of the patient can be estimated using a neural network, for example, based on a set of vectors between points on the body and a temperature sensor.

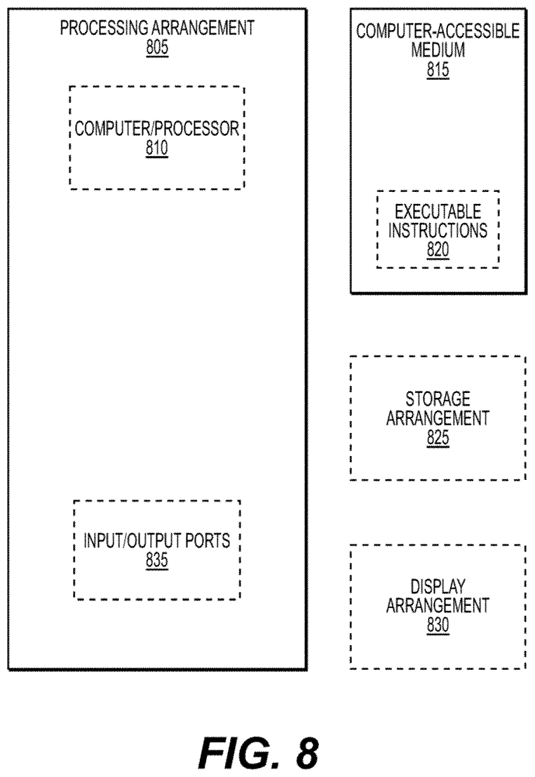

FIG. 8 shows a block diagram of an exemplary embodiment of a system according to the present disclosure. For example, exemplary procedures in accordance with the present disclosure described herein can be performed by a processing arrangement and/or a computing arrangement (e.g., computer hardware arrangement or a hardware computing arrangement) 805. Such processing/computing arrangement 805 can be, for example entirely or a part of, or include, but not limited to, a computer/processor 810 that can include, for example one or more microprocessors, and use instructions stored on a computer-accessible medium (e.g., RAM, ROM, hard drive, or other storage device).

As shown in FIG. 8, for example a computer-accessible medium 815 (e.g., as described herein above, a storage device such as a hard disk, floppy disk, memory stick, CD-ROM, RAM, ROM, etc., or a collection thereof) can be provided (e.g., in communication with the processing arrangement 805). The computer-accessible medium 815 can contain executable instructions 820 thereon. In addition or alternatively, a storage arrangement 825 can be provided separately from the computer-accessible medium 815, which can provide the instructions to the processing arrangement 805 so as to configure the processing arrangement to execute certain exemplary procedures, processes, and methods, as described herein above, for example.

Further, the exemplary processing arrangement 805 can be provided with or include an input/output ports 835, which can include, for example a wired network, a wireless network, the internet, an intranet, a data collection probe, a sensor, etc. As shown in FIG. 8, the exemplary processing arrangement 805 can be in communication with an exemplary display arrangement 830, which, according to certain exemplary embodiments of the present disclosure, can be a touch-screen configured for inputting information to the processing arrangement in addition to outputting information from the processing arrangement, for example. Further, the exemplary display arrangement 830 and/or a storage arrangement 825 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used. synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

1. A non-transitory computer-accessible medium having stored thereon computer-executable instructions for estimating a temperature on a portion of a body of at least one anatomical structure, wherein, when a hardware computing arrangement executes the instructions, the hardware computing arrangement is configured to perform procedures comprising:
receiving a plurality of magnetic resonance (MR) images for the at least one anatomical structure;
segmenting the MR images into a plurality of tissue types;
mapping the tissue types to at least one tissue property; and
estimating the temperature on the body of the at least one patient using a neural network.

2. The computer-accessible medium of claim 1, wherein the at least one tissue property includes at least one of a conductivity, a permittivity or a density.

3. The computer-accessible medium of claim 1, wherein the density is a mass cell density.

4. The computer-accessible medium of claim 1, wherein the neural network is a single neural network.

5. The computer-accessible medium of claim 1, wherein the hardware computing arrangement is configured to estimate the temperature based on a set of vectors between points on the body and a temperature sensor.

6. The computer-accessible medium of claim 1, wherein each of the vectors corresponds to a tissue thermal profile for each respective point.

7. The computer-accessible medium of claim 6, wherein the hardware computing arrangement is further configured to map the temperature at each respective point.

8. The computer-accessible medium of claim 7, wherein the hardware computing arrangement is configured to map the temperature at each respective point using the neural network.

9. The computer-accessible medium of claim 7, wherein the hardware computing arrangement is configured to map the temperature at each point using a Euclidean distance between each respective point and a temperature sensor.

10. The computer-accessible medium of claim 1, wherein the portion of the body is on a surface of the at least one anatomical structure.

11. The computer-accessible medium of claim 1, wherein the portion of the body is internal to the at least one anatomical structure.

12. The computer-accessible medium of claim 1, wherein the tissue types include at least one of (i) Fat, (ii) Grey Matter, (iii) Bone, (iv) Muscle, or (iv) Cerebrospinal Fluid.

13. The computer-accessible medium of claim 1, wherein the hardware computing arrangement is further configured to train the neural network.

14. The computer-accessible medium of claim 13, wherein the hardware computing arrangement is configured to train the neural network by segmenting the tissue types of at least one further anatomical structure.

15. The computer-accessible medium of claim 14, wherein the tissue types include at least one of (i) Fat, (ii) Grey Matter, (iii) Bone, (iv) Muscle, of (iv) Cerebrospinal Fluid.

16. The computer-accessible medium of claim 13, wherein the hardware computing arrangement is configured to train the neural network by varying a number of hidden nodes in the neural network.

17. The computer-accessible medium of claim 1, wherein the neural network includes (i) three layers, and (ii) a Rectified linear Unit activation function.

18. The computer-accessible medium of claim 1, wherein the at least one anatomical structure is a brain of a patient, and wherein the MR images are brain slices of the brain of the patient.

19. A method for estimating a temperature on a portion of a body of at least one anatomical structure, comprising:
receiving a plurality of magnetic resonance (MR) images for the at least one anatomical structure;
segmenting the MR images into a plurality of tissue types;
mapping the tissue types to at least one tissue property; and
using a hardware computing arrangement, estimating the temperature on the body of the at least one patient using a neural network.

20-36 (canceled)

37. A system for estimating a temperature on a portion of a body of at least one anatomical structure, comprising:
a hardware computing arrangement configured to:
receive a plurality of magnetic resonance (MR) images for the at least one anatomical structure;
segment the MR images into a plurality of tissue types;
map the tissue types to at least one tissue property; and
estimate the temperature on the body of the at least one patient using a neural network.

38-54. (canceled)
